(19) United States
(12) Patent Application Publication
(10) Pub. No.: US 2025/0169972 A1
(43) Pub. Date: May 29, 2025

(54) RECOLLAPSIBLE AND RECAPTURABLE DEVICES AND METHODS OF USE

(71) Applicant: Cardiac Dimensions Pty. Ltd., Kirkland, WA (US)

(72) Inventors: Timothy JOHNSON, Kent, WA (US); Robert CALCOTE, Kirkland, WA (US)

(21) Appl. No.: 18/840,878

(22) PCT Filed: Feb. 24, 2023
(86) PCT No.: PCT/US2023/063206

§ 371 Date: Aug. 22, 2024

Related U.S. Application Data
(60) Provisional application No. 63/268,581, filed on Feb. 25, 2022.

(51) Int. Cl. A61F 2/915; A61F 2/95

(52) U.S. Cl. CPC A61F 2/915; A61F 2/95; A61F 2002/91575; A61F 2002/9511

(57) ABSTRACT

Expandable stent anchors that are adapted and configured to be recollapsed after expansion if needed. The disclosure is related to an implantable device adapted to reduce mitral valve regurgitation, comprising a proximal stent anchor, a distal stent anchor, and a connector extending from the proximal stent anchor to the distal stent anchor. The stent anchors may include one or more ramped regions that facilitate recollapse of the stent anchors after expansion. The proximal anchor includes a coupler that is sized and configured to be coupled to a delivery system for delivery of the implantable device, wherein the ramped region of the proximal stent anchor extends distally and radially relative to the coupler.

### CROSS REFERENCE TO RELATED APPLICATIONS

This application claims priority to U.S. Provisional Application No. 63/268,581, filed Feb. 25, 2022, which is incorporated by reference herein in its entirety for all purposes.

### INCORPORATION BY REFERENCE

All publications and patent applications mentioned in this specification are herein incorporated by reference herein for all purposes to the same extent as if each individual publication or patent application was specifically and individually indicated to be incorporated by reference.

### BACKGROUND

Expandable medical devices, such as stents, have been developed to be delivered in a collapsed or delivery configuration, and expanded at a target site within a patient, such as within the vasculature of the patient. Expandable medical devices are needed that are adapted to both reliably anchor in place after expansion, and be recollapsed and/or repositioned if necessary.

### SUMMARY OF THE DISCLOSURE

One aspect of the disclosure is an implantable device adapted to reduce mitral valve regurgitation, comprising: a proximal stent anchor, a distal stent anchor, and a connector extending from the proximal stent anchor to the distal stent anchor, the proximal stent anchor including a proximal region including a ramped region having, in a side view, a ramped configuration, the proximal region of the proximal stent anchor including a proximal end of the device, the proximal region of the proximal anchor further including a coupler that is sized and configured to be coupled to a delivery system for delivery of the implantable device, the coupler disposed at a proximal end of the device, and wherein the ramped region of the proximal stent anchor extends distally and radially relative to the coupler, and the distal stent anchor including a proximal region including a ramped region having, in a side view, a ramped configuration, the ramped region of the distal stent anchor extending distally and radially from a distal end of the connector.

In this aspect, the proximal stent anchor, the connector and the distal stent anchor are optionally monolithically formed from a tubular member, optionally a nitinol tube.

In this aspect, the coupler and the connector are optionally aligned along a common axis that extends through a radial periphery of the proximal stent anchor and a radial periphery of the distal stent anchor. A common axis is optionally curved.

In this aspect, the coupler is optionally disposed at a radial periphery of the proximal stent anchor.

In this aspect, the connector is optionally positioned such that proximal stent anchor and the distal stent anchor expand radially away from the connector during expansion.

In this aspect, the coupler is optionally positioned such that the proximal stent anchor expands radially away from the coupler during expansion.

In this aspect, the connector optionally extends distally from a radial periphery of the proximal stent anchor, and optionally extends proximally from a radial periphery of the distal stent anchor.

In this aspect, the ramped regions optionally each have, in an end view of the respective stent anchor, an annular configuration.

In this aspect, the proximal stent anchor optionally further includes a distal region including a distal ramped region having, in the side view, a ramped configuration in which the distal ramped region extends distally and radially inward.

In this aspect, the distal stent anchor optionally further includes a distal region including a distal ramped region having, in a side view, a ramped configuration, the distal ramped region of the distal stent anchor extending distally and radially inward.

An additional aspect of this disclosure is an expandable stent anchor (optionally self-expandable) adapted to be recollapsed and recaptured after expansion. In this aspect, the stent anchor may include any other feature or description of any other stents or stent anchors herein.

In this aspect, the stent anchor optionally includes a proximal region including a ramped region having, in a side view, a ramped configuration. The proximal region optionally further includes a coupler that is sized and configured to be releasably coupled to a delivery system for delivery of the stent anchor, the coupler disposed at a proximal end of the stent, and wherein the ramped region extends distally and radially relative to the coupler.

In this aspect, a coupler is optionally disposed at a radial periphery of the stent.

In this aspect, a distal end of a ramped region are optionally disposed at a second radial periphery of the stent.

In this aspect, a coupler and a distal end of a ramped region are optionally 180 degrees circumferentially apart from one another.

In this aspect, a coupler optionally comprises a bend in an elongate section of the stent, wherein the elongate section includes first and section sections extending generally distally from a proximal end of the coupler. A coupler optionally comprises an aperture therethrough.

In this aspect, the stent is optionally monolithic structure formed from a single piece of starting material, optionally laser cutting a tube, which may facilitate the manufacturing process.

In this aspect, a ramped stent region optionally has, in an end view of the stent, an annular configuration.

### BRIEF DESCRIPTION OF THE DRAWINGS

FIG. 1A is a side view of an exemplary expandable and recollapsible stent anchor.

FIG. 1B is a top view of an exemplary expandable and recollapsible stent anchor.

FIG. 1C is an end view of an exemplary expandable and recollapsible stent anchor.

FIG. 1D is a side view of an exemplary expandable and recollapsible stent anchor during a recollapsing step.

FIG. 2A is an example of a ramped configuration of a stent anchor.

FIG. 2B is an example of a ramped configuration of a stent anchor.

FIG. 2C is an example of a ramped configuration of a stent anchor.

FIG. 3 is a close up perspective view of an exemplary releasable coupling between a stent anchor and an exemplary delivery system.

FIG. 4 is an exemplary end region of a stent anchor that has a cylindrical configuration.

FIG. 5A is a side view of an exemplary tissue reshaping device including a plurality of expandable and recollapsible stent anchors.

FIG. 5B is a top view of an exemplary tissue reshaping device including a plurality of expandable and recollapsible stent anchors.

FIG. 5C is an end view of a single stent anchor.

FIG. 6 illustrates an exemplary location for implanting tissue reshaping devices comprising a plurality of expandable and recollapsible stent anchors.

FIG. 7 is a side view of an exemplary expandable and recollapsible stent anchor.

FIG. 8 is a side view of an exemplary expandable and recollapsible stent anchor.

FIG. 9 is a side view of an exemplary tissue reshaping device including a plurality of expandable and recollapsible stent anchors.

### DETAILED DESCRIPTION

This disclosure is related to recollapsible and recapturable medical devices, and particularly to recollapsible and recapturable stent devices that include one or more stent anchors. Expandable stents may be deployed and find use in a variety of target anatomical locations within a patient. Stents herein may be referred to as stent anchors, and vice versa.

In some merely exemplary applications, the recollapsible stent anchors and stent devices herein may be configured for use in a structured heart context generally. For example only, the recollapsible stent anchors herein may be sized and configured for deployment in the great cardiac vein, and may be used for heart valve repair, such as reshaping a mitral valve annulus to treat mitral valve regurgitation.

In some exemplary applications, a medical device may include a plurality of expandable stent anchors (e.g., first and second) with an axially extending connector therebetween, wherein at least one of the plurality of expandable stent anchors may be configured as any of the recollapsible stent anchors herein. For example, any of the disclosure from any of the following references may be incorporated into any of the examples and embodiments herein, including any and all disclosure related to methods of deploying and using a mitral valve annulus reshaping device to reshape a mitral valve annulus to reduce mitral valve regurgitation: U.S. Pat. Nos. 6,976,995; 6,960,229; 7,351,260; 7,316,708; 7,837,729; 8,182,529; and U.S. Pat. App. No. 2008/0015679.

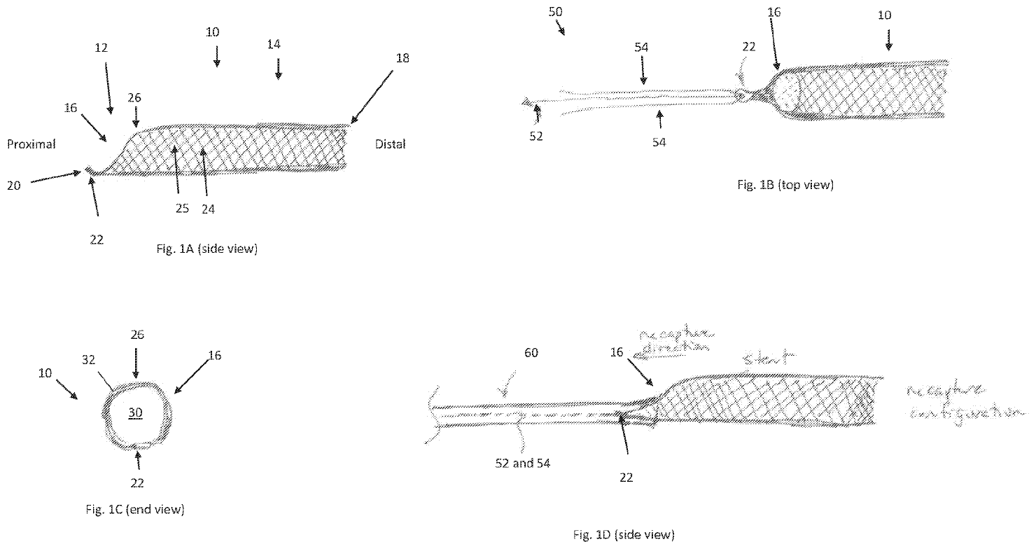

FIG. 1A illustrates a side view of an exemplary recapturable and recollapsible stent anchor 10 in an expanded configuration after being released from a delivery system (not shown), which extends from proximal end 20 to distal end 18. Stent anchor 10 includes a proximal region 12, which includes ramped region 16. Ramped region 16 has, in the side view as shown, a ramped configuration. Proximal region 12 includes proximal end 20 of stent anchor 10. Proximal region 12 further includes a coupler 22 that is sized, configured and positioned to be releasably coupled to a delivery system to facilitate delivery of stent 10 to a target location within a patient. Coupler 22 is disposed at and defines proximal end 20 of stent anchor 10, and ramped region 16 extends distally and radially relative to coupler 22, as shown. Stent 10 also includes distal region 14, including stent distal end 18. Distal region 14 extends distally from proximal region 12, and may have a cylindrical configuration, as shown in the example of FIG. 1A. Proximal region 12 and distal region 14 are considered part of the same expandable and collapsible structure.

Stent 10 includes a plurality of elongate members 24, which generally define the body of stent anchor 10 and also define a plurality of open cells 25 (only one cell labeled). Open cells are considered open space between the plurality of elongate members 24. Elongate members 24 may be formed from one or more elements, such as a single element or a plurality of elements. In a merely exemplary embodiment, stent anchor 10 is formed by laser cutting an elastic tubular member, such as a nitinol tube, such that elongate members 24 are part of a monolithic or unitary structure, wherein after the laser cutting formation, elongate members 24 define open cells 25. In other embodiments, elongate members may be formed using other techniques, such as braiding one or more braidable materials, such as braidable filaments.

Stent anchor 10 is adapted to be at least partially self-expandable when released or deployed from a delivery device, and may be adapted to be fully self-expandable to an expanded configuration (e.g., as shown in FIG. 1A). Stent anchor 10 is also adapted to be recollapsed and recaptured after it has been expanded to the expanded configuration. Ramped region 16, which has a proximal end with a ramped configuration (as shown), is shaped, sized and positioned to facilitate recollapse and recapture into a distal end of a delivery device, if needed, as discussed below.

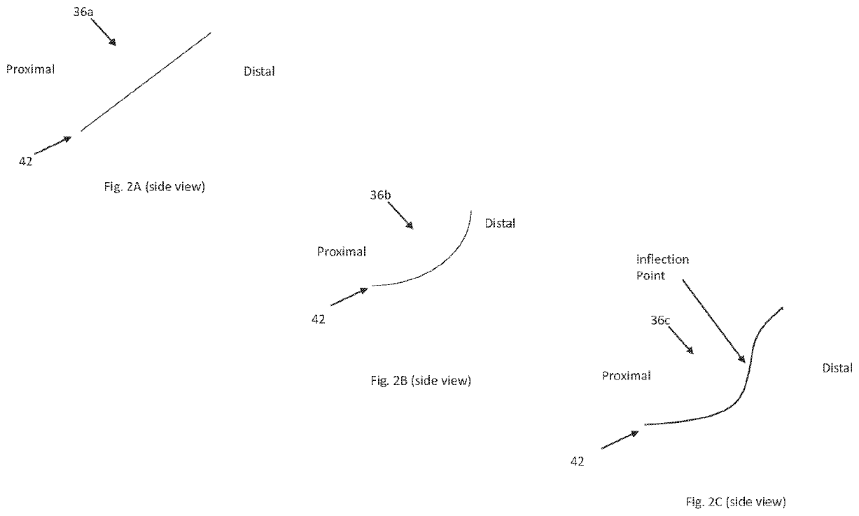

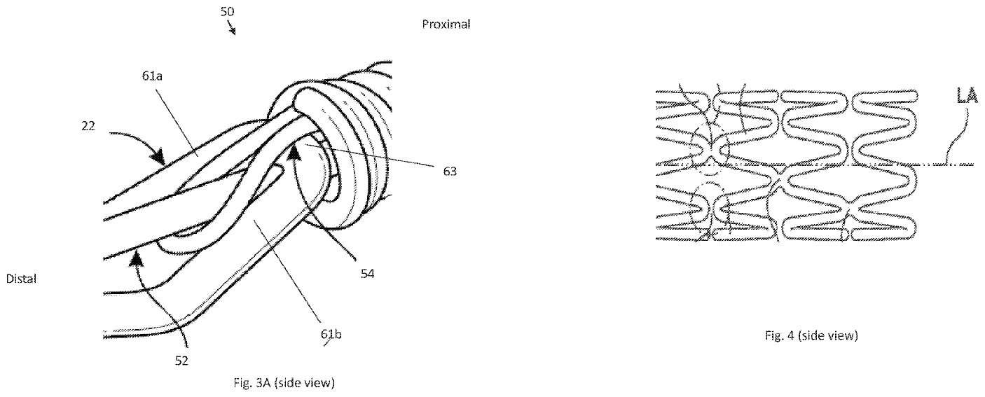

FIGS. 2A, 2B and 2C illustrate side views of configurations of merely exemplary ramped regions 36a, 36b an 36c respectively (the entire stent not shown for clarity). FIG. 2A illustrates ramped region 36a, which has a flat configuration with a constant slope along its length from a proximal end of ramped region 36a to a distal end of ramped region 36a. FIG. 2B illustrates ramped region 36b, which includes at least one curved section, and in this example is curved along its length from a proximal end of ramped region 36b to a distal end of ramped region 36b. FIG. 2C illustrates ramped region 36c which is curved, and which includes an inflection point, as shown. The relative position of couplers 42 is also illustrated in FIGS. 2A-2C.

FIG. 1C illustrates an end view of stent anchor 10, looking proximal to distal. Stent anchor 10 defines lumen 30, which are generally defined by the plurality of elongate members 24. In this example, in the end view, stent anchor 10 and lumen 30 have generally circular configurations, as shown. Stent anchor 10 and lumen 30 may have slightly different configurations, in the end view, however, such as an oval (e.g., having a width dimension greater than a height dimension).

As shown in FIG. 1C, coupler 22 is disposed 180 degrees circumferentially away from distal end 26 of the ramped region. It is understood that 180 degrees in this context includes slight variations from exactly 180 degrees such as +/−10 degrees (e.g., 170 degrees). As is also shown in FIG. 1C, stent anchor 10, including ramped region 16, has a circular, or annular configuration. In this example, ramped region 16 includes surface 32, which extends distally and radially relative to coupler 22, and which also extends circumferentially around ramped region 16 to form the annular configuration in the end view of FIG. 1C. Surface 32 may be a smooth surface to facilitate the recollapse of the stent, which is described below. Surface 32, in the end view, defines a significant portion of the radial periphery of the stent. Coupler 22 is disposed at a radial periphery of the stent, as shown in FIGS. 1A, 1C and 1D. Distal end 26 of ramped region 16 is disposed at a second radial periphery of the stent anchor, as shown, and coupler 22 and distal end 26 of ramped region 16 are, in this embodiment, 180 degrees circumferentially apart or offset from one another.

The stent anchors herein are adapted to be, if necessary, recollapsed after expansion and to be recaptured within a delivery device. This can be beneficial or necessary if one or more stents of a device needs to be moved to a new location, for example, or if the device needs to be removed from the patient after at least a first stent has been deployed from a delivery device. The stent anchors include a coupler 22 at a proximal end 20 of the stent anchor, which allows the stent to be releasably coupled or connected to a delivery system during delivery and until the stent is to be released from the delivery system inside the patient, and which also facilitates recollapse of the stent anchor. In some variations, a delivery system (or secondary device) may reconnect with coupler 22 even after a delivery system is detached from coupler 22, such as if the stent anchor is to be recollapsed, retrieved, and removed from the patient.

FIG. 1B illustrates a top view of stent anchor 10 in an expanded configuration and connected or coupled to a portion of delivery system 50. It is understood that delivery system 50 is exemplary, and other types of delivery systems may be used to delivery and recapture the stents herein. FIG. 3A also illustrates a close-up perspective view of an exemplary implementation of a coupling mechanism between stent coupler 22 and delivery system 50. In this example, delivery system 50 may include lock wire 52, which extends through coupler 22, as shown in FIGS. 1B, 1D and 3A. Delivery system 50 also includes tether wire 54, which extends around lock wire 52 as shown in FIG. 3A. To release stent anchor 10, lock wire 52 is retracted proximally away from stent coupler 22, which releases stent anchor 10 from delivery system 50. Additional details of this merely exemplary uncoupling process are described in publications incorporated by reference herein, the entire disclosures of which are incorporated by reference herein, including into this particular example, in their entireties.

Prior to uncoupling from the delivery system, however, it may be desirable or necessary to recollapse and recapture stent anchor 10, such as for repositioning, or even removal from the patient. FIG. 1D illustrates a process in which proximal region 16 of stent anchor 10 is being collapsed radially downward and into tubular member 60, which may be a catheter that is also part of delivery system 50 in which stent anchor 10 is initially delivered. Tubular member 60 may optionally be adapted to flare slightly at its distal end, as shown, which may help facilitate recollapse of stent anchor 10. To recollapse stent, stent anchor 10 may be pulled proximally, tubular member 60 may be advanced distally, or a combination of both types of relative movement. Proximal and distal movement in this context are examples of axial movement herein. The ramped configuration of ramped region 16, which extends distally and radially relative to coupler 22, allows the distal end of tubular member 60 to contact and apply a force on ramped region 16, which causes the collapse of the ramped region 16, which thereby causes collapse of the stent distal to ramped region 16. The ramped configuration of ramped region 16 thereby facilitates partial or complete recollapse and recapture of the entire stent anchor 10, even after the stent has been fully expanded. Once recaptured inside tubular member 60, the stent may be repositioned or even retrieved from the patient if desired or needed.

The stent couplers herein (e.g., coupler 22) may have a variety of configurations that allows them to be coupled to a variety of delivery systems. Exemplary coupler 22 includes bend 63 in an elongate section of the stent, as labeled in FIG. 3A, in which first and section sections 61a and 61b extend generally distally from a proximal end of coupler 22. In some examples, couplers herein may have a general “V” or “U” configuration, or other similar configuration. Couplers herein are shown as having and defining an aperture, or window therethrough, which facilitates coupling to one or more delivery system components, examples of which are described herein.

FIG. 4 illustrates a merely exemplary distal region of an exemplary stent (proximal region not shown), which illustrates a merely exemplary laser cut pattern, and which is described in more detail in U.S. Pat. No. 9,114,032, all of which is incorporated by reference herein in its entirety in this context. The distal region as shown in FIG. 4 may be incorporated into or be any of the distal regions of any of the stents herein, unless indicated to the contrary herein.

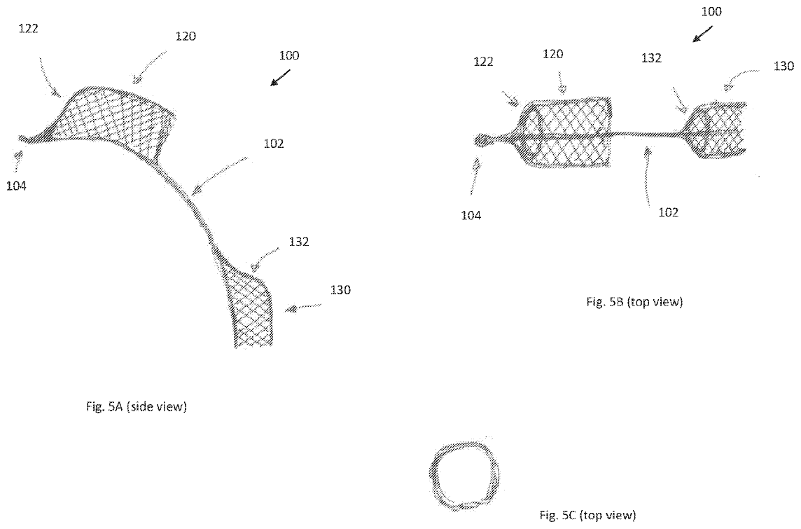

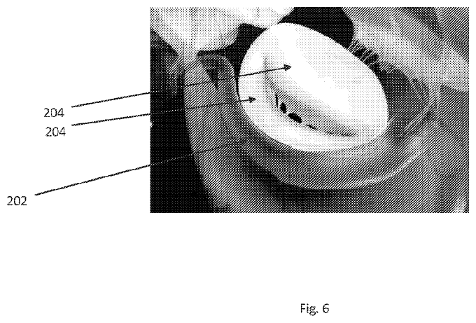

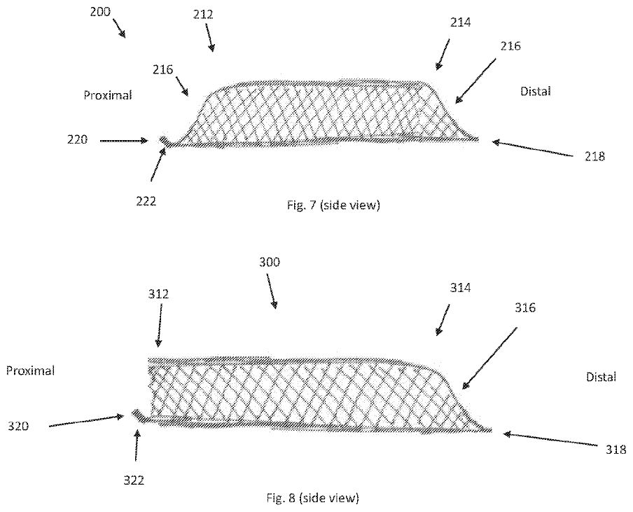

FIGS. 5A, 5B and 5C illustrate an exemplary embodiment of a dual-anchored device that includes one or more recollapsible and recapturable stents. The stents in FIGS. 5A-5C are understood to optionally include any of the features of any of the stents described with reference to FIGS. 1A-4 described herein. The embodiment in FIGS. 5A-5C may also incorporate by reference any general description of any of the references incorporated by reference herein, such as a proximal anchor that is larger than a distal anchor when the anchors are expanded. In a merely exemplary use, the device in FIGS. 5A-5C may be deployed in a great cardiac vein (optionally within a coronary sinus, optionally completely within a coronary sinus) and used to reshape mitral valve annulus tissue to reduce mitral valve regurgitation, the disclosure of which is incorporated by reference herein from any of the references that have been incorporated by reference herein. FIG. 6 illustrates a coronary sinus 202, and mitral valve leaflets 204, wherein the device from FIGS. 5A-5C may be deployed in the coronary sinus as described in any of the materials incorporated by reference herein and used to reduce regurgitation between leaflets 204.

FIG. 5A illustrates a side view of mitral valve annulus reshaping device 100, wherein proximal stent anchor 120 and distal stent anchor 130 are both in expanded configurations (optionally completely self-expandable) and wherein device 100 has been released or uncoupled from a delivery system. In this example, proximal stent anchor 120 and distal stent anchor 130 are stent anchors, and may include any or all of the features of stent 10. For example, proximal stent anchor 120 includes a proximal region that includes ramped region 122, which has a ramped configuration as shown. Similarly, distal stent anchor 130 includes a proximal region that includes ramped region 132, which has a ramped configuration as shown. Device 100 includes connector 102 extending from proximal anchor 120 to distal anchor 130. As shown, connector 102 extends from radial peripheries of the proximal and distal anchors. Connectors has a slight bend formed therein, as shown in FIG. 5A.

FIG. 5B illustrates a top view of device 100, with connector 102 having been bent so that it is in a straightened configuration (FIG. 5A shows an at-rest, as-manufactured, or unconstrained device 100). Optionally, the configuration in FIG. 5B is an alternative to device 100 in FIG. 5A, in which connector 102 has an at-rest, as-manufactured, unconstrained straight configuration. Device 100 may optionally be laser cut from a single tubular starting material, such as an elastic tubular member (e.g., a nitinol tube), and thus device 100 may be monolithic or unitary. Connector 102 may thus be unitary and monolithically formed with the stent anchors 120 and 130. Connector 102 may have a rectangular cross-sectional configuration, with all sides having a slight curvature if cut from a tubular material. Connector 102 has a fixed length between the anchors, with a proximal end and a distal end that do not move axially relative to the anchors. This is particularly the case when the connectors and anchors are monolithically formed or unitary.

Coupler 104 may be any of the couplers described herein (e.g., coupler 22), and is adapted to be releasably coupled to a delivery system. Device 100 may be releasably coupled to and delivered with any of the delivery systems herein, such as delivery system 50 that is described herein.

In the example of the device shown in FIGS. 5A-5C, both of the expandable and recollapsible anchors have ramped proximal regions, which are adapted to be recollapsed and recaptured within a tubular member in needed. Distal anchor 130 may be recaptured after it has been expanded (in a coronary sinus, for example) even before connector 102 and proximal anchor 120 are deployed outside of the delivery device (e.g., tubular member 60). Additionally, after proximal anchor 120 expands (e.g., fully self-expandable), proximal anchor 120 may be recaptured and recollapsed by causing the distal end of a tubular member to apply a force to the surface of ramped region 122, which facilitates collapse of the ramped region and distal region of the proximal anchor, as is described above. If the entire device 100 is to be recaptured, the tubular member (e.g., tubular member 60) may be axially moved (relative motion) over the connector and used to similarly recollapse distal anchor 130 by applying a force from the distal end of the tubular member on the ramped region 132 of the distal anchor, which causes collapse of the ramped region 132 and eventually the entire distal anchor 130. Device 100 may then be repositioned or removed within the delivery device.

While the disclosure above includes stent anchors that comprise proximal regions that include ramped regions (with ramped configurations), any of the stent anchors herein may additionally or alternatively include distal regions that include ramped regions. In any of these embodiments, a distal region of the stent anchor may comprise a distal ramped region that tapers radially inward (or downward) toward a distal end of the stent anchor, examples of which are shown in exemplary FIGS. 7-9. Any of the distal ramped regions herein may include any suitable ramped region feature and/or configuration herein even if not expressly included in the disclosure herein, and even if the feature is described with respect to a proximal ramped region or configuration. For example only, any distal ramped region herein may have, in an end view (looking from distal to proximal), an annular configuration, such as is shown in the end views of the exemplary proximal ramped regions in FIGS. 1C and 5C. Additionally, and for example only, any distal ramped region herein may include a ramped surface, and which may include any suitable feature of ramped surface 32 shown in FIG. 1C.

FIG. 7 illustrates a side view of an exemplary recapturable and recollapsible stent anchor 200 in an expanded configuration and which extends from proximal end 220 to distal end 218. Stent anchor 200 may include any suitable feature of any stent herein, including stent 10 in FIGS. 1A-ID. Stent anchor 200 includes proximal region 212, which includes ramped region 216 with a ramped configuration, which may include any suitable feature of any proximal ramped region herein (e.g., ramped region 16). Proximal region 212 also includes proximal end 220 of stent anchor 200. Proximal region 212 further includes a coupler 222 that is sized and configured to be releasably coupled to a delivery system to facilitate delivery of stent anchor 200 to a target location within the patient. Stent 200 also includes distal region 214, which includes stent distal end 218. Distal region 214 extends distally from proximal region 212, and includes distal ramped region 216, which as shown has a ramped configuration that tapers radially inward or downward towards and relative to distal end 218.

Stent anchor 200 includes a plurality of elongate members as shown, which generally define the body of stent anchor 200 and define a plurality of cells, and any and all disclosure herein related to elongate members is incorporated by reference into stent anchor 200. The elongate members may be formed from one or more elements, such as a single element or a plurality of elements. Stent anchor 200 may be formed by laser cutting an elastic tubular member, such as a nitinol tube, such that the elongate members are part of a monolithic or unitary structure. In other embodiments, elongate members may be formed using other techniques, such as braiding one or more braidable materials, such as braidable filaments.

Stent anchor 200 is adapted to be at least partially self-expandable when released from a delivery device, and may be adapted to be fully self-expandable to an expanded configuration (e.g., as shown in FIG. 7). Stent anchor 200 is also adapted to be recollapsed and recaptured after expanding to the expanded configuration. One or more of proximal ramped region 216 and distal ramped region 216 may be configured to facilitate (or at least help with) the collapse (e.g., the recollapse) and recapture into a delivery device, as discussed herein.

The ramped configuration of distal ramped region 216 (and other distal ramped regions herein) may help the stent anchor more easily collapse to a collapsed delivery configuration and/or it may also help the stent be recollapsed if necessary after partial or full expansion, which is described in more detail herein. For example, distal ramped regions herein may help reduce the collapse force necessary to collapse the stent anchor.

FIG. 8 illustrates a side view of an exemplary recapturable and recollapsible stent anchor 300 in an expanded configuration after being released from a delivery system (not shown), which extends from proximal end 320 to distal end 318. Stent anchor 300 may include any suitable feature of any stent herein, including stent 10 in FIGS. 1A-1D. Stent anchor 300 includes a proximal region 312, which optionally does not have a ramped configuration as do other stent anchors herein, and which may be considered to have a generally cylindrical configuration. Proximal region 312 includes proximal end 320 of stent anchor 300. Proximal region 312 further includes a coupler 322 that is sized and configured to be releasably coupled to a delivery system to facilitate delivery of stent anchor 300 to a target location within the patient. Stent 300 also includes distal region 314, which includes stent distal end 318. Distal region 314 extends distally from proximal region 312, and includes distal ramped region 316, which as shown has a ramped configuration and tapers radially inward or downward towards and relative to stent distal end 318. Stent anchor 300 includes a plurality of elongate members as shown, which generally define the body of stent 300 and define a plurality of cells. The elongate members may be formed from one or more elements, such as a single element or a plurality of elements. Stent anchor 300 may be formed by laser cutting an elastic tubular member, such as a nitinol tube, such that the elongate members are part of a monolithic or unitary structure. In other embodiments, elongate members may be formed using other techniques, such as braiding one or more braidable materials, such as braidable filaments.

Stent anchor 300 is adapted to be at least partially self-expandable when released from a delivery device, and may be adapted to be fully self-expandable to an expanded configuration (e.g., as shown in FIG. 8). Stent 300 is also adapted to be recollapsed and recaptured after expanding to the expanded configuration. Distal ramped region 316 may be configured to help facilitate the collapse (e.g., the recollapse) and recapture into a delivery device, as described herein.

The ramped configuration of distal ramped region 316 (and other distal ramped regions herein) may help the stent anchor more easily collapse to a collapsed delivery configuration, while it may also help the stent be recollapsed if necessary after partial or full expansion, which is described in more detail herein.

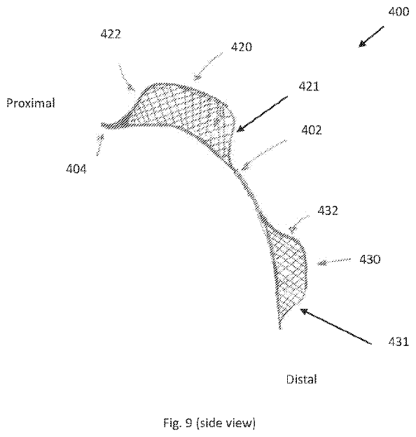

FIG. 9 illustrates a side view of mitral valve annulus reshaping device 400, which may include any suitable feature of device 100 shown in FIGS. 5A-5C, wherein proximal stent anchor 420 and distal stent anchor 430 are both shown in expanded configurations (optionally completely self-expandable). In this example, proximal anchor 420 and distal anchor 430 are stent anchors, and may each include any or all of the features of any of the stents herein, including but not limited to stents 200 and 300. For example, proximal stent anchor 420 includes a proximal region that includes ramped region 122 and a distal region that includes ramped region 421, each of which has a ramped configuration as shown. Similarly, distal stent anchor 430 includes a proximal region that includes ramped region 432 and a distal region that includes ramped region 431, each of which has a ramped configuration as shown. Device 400 includes connector 402 extending from proximal anchor 420 to distal anchor 430. As shown, connector 402 extends from radial peripheries of the anchors. Device 400 may be monolithically formed, or unitary, such as formed from a tubular member, which is described herein.

While not shown, a further alternative to device 400 may include proximal and distal anchors, wherein each anchor may comprise distal ramped regions but not proximal ramped regions. For example, in this alternative, both distal and proximal anchors may be the same as or similar to the anchor shown in FIG. 8.

In a further alternative to device 400, one of the anchors (e.g., the proximal anchor or the distal anchor) may comprise a proximal ramped region but not a distal ramped region, while the other anchor (e.g., the proximal anchor or the distal anchor) may comprise a distal ramped region but not a proximal ramped region.

In a further alternative to device 400, only one of the anchors (e.g., the proximal anchor or the distal anchor) may comprise a ramped region (proximal ramped region and/or distal ramped region), while the other anchor may not comprise a ramped region (i.e., neither proximal nor distal).

Unless indicated to the contrary herein, any number of features from one example or embodiment may be combined or incorporated with any other example or embodiment.

1. An implantable device adapted to reduce mitral valve regurgitation, comprising:
a proximal stent anchor, a distal stent anchor, and a connector extending from the proximal stent anchor to the distal stent anchor,
the proximal stent anchor including a proximal region including a ramped region having, in a side view, a ramped configuration, the proximal region of the proximal stent anchor including a proximal end of the device,
the proximal region of the proximal anchor further including a coupler that is sized and configured to be coupled to a delivery system for delivery of the implantable device, the coupler disposed at a proximal end of the device, and wherein the ramped region of the proximal stent anchor extends distally and radially relative to the coupler,
the distal stent anchor including a proximal region including a ramped region having, in a side view, a ramped configuration, the ramped region of the distal stent anchor extending distally and radially from a distal end of the connector.

2. The device of claim 1, wherein the proximal stent anchor, the connector and the distal stent anchor are monolithically formed from a tubular member.

3. The device of claim 2, wherein the proximal stent anchor, the connector and the distal stent anchor are monolithically formed from a nitinol tubular member.

4. The device of claim 1, wherein the coupler and the connector are aligned along a common axis that extends through a radial periphery of the proximal stent anchor and a radial periphery of the distal stent anchor.

5. The device of claim 4, wherein the common axis is curved (e.g., as shown in FIG. 5A).

6. The device of claim 1, wherein the coupler is disposed at a radial periphery of the proximal stent anchor.

7. The device of claim 1, wherein the connector is positioned such that proximal stent anchor and the distal stent anchor expand radially away from the connector during expansion.

8. The device of claim 1, wherein the coupler is positioned such that the proximal stent anchor expands radially away from the coupler during expansion.

9. The device of claim 1, wherein the connector extends distally from a radial periphery of the proximal stent anchor, and extends proximally from a radial periphery of the distal stent anchor.

10. The device of claim 1, wherein the ramped regions each have, in an end view of the respective stent anchor, an annular configuration.

11. The device of claim 1, wherein the proximal stent anchor further includes a distal region including a distal ramped region having, in the side view, a ramped configuration in which the distal ramped region extends distally and radially inward.

12. The device of claim 1, wherein the distal stent anchor further includes a distal region including a distal ramped region having, in a side view, a ramped configuration, the distal ramped region of the distal anchor extending distally and radially inward.

13. A method of positioning a recollapsible tissue reshaping device, comprising:
delivering a tissue reshaping device in a collapsed configuration within a delivery device, wherein the tissue reshaping device includes a coupler that is releasably coupled to a delivery system coupler;
deploying a distal stent anchor of the tissue reshaping device from the delivery device to allow the distal stent anchor to self-expand to an expanded configuration in which the distal stent anchor has a proximal region that has a ramped region with a ramped configuration, wherein a connector of the tissue reshaping device extends proximally from the ramped region;
deploying the connector from the delivery device; and
deploying a proximal stent anchor of the tissue reshaping device from the delivery device to allow the proximal stent anchor to self-expand to an expanded configuration in which the proximal stent anchor has a proximal region that has a ramped region with a ramped configuration, wherein a ramped region of the proximal stent anchor extends distally and radially from the coupler.

14. The method of claim 13, further comprising recollapsing the distal stent anchor at a time subsequent to allowing the distal stent anchor to self-expand.

15. The method of claim 13, further comprising recollapsing the proximal stent anchor at a time subsequent to allowing the proximal stent anchor to self-expand.

16. The method of claim 15, wherein recollapsing the proximal stent anchor comprises causing relative axial movement between the proximal stent anchor and the delivery device, wherein the relative axial movement causes a distal end of the delivery device to apply a force on the ramped region of the proximal stent anchor, which causes collapse of the ramped region of the proximal stent anchor.

17. The method of claim 16, wherein recollapsing the proximal stent anchor occurs while the coupler is releasably coupled to a delivery system coupler.

18. The method of claim 13, further comprising releasing the delivery system coupler from the coupler to release the device from the delivery system coupler.

19. The method of claim 13, further comprising pulling proximally on the expanded distal stent anchor to reshape mitral valve annulus tissue and reduce mitral valve regurgitation.

20. The method of claim 19, wherein deploying the proximal stent anchor occurs at a time subsequent to pulling proximally on the expanded distal stent anchor.

21. The method of claim 13, wherein deploying the distal stent anchor allows the distal stent anchor to self-expand to the expanded configuration in which the distal stent anchor has a distal region that has a distal ramped region with a ramped configuration that extends distally and radially inward.

22.-44. (canceled)

45. A method of positioning a recollapsible stent anchor, comprising:
delivering a recollapsible stent anchor in a collapsed configuration within a delivery device, wherein the stent anchor includes a coupler that is releasably coupled to a delivery system coupler,
deploying the stent anchor from the delivery device to allow the stent to self-expand to an expanded configuration in which the stent anchor has a proximal region that has a ramped region with a ramped configuration, the proximal region including the coupler, and wherein the ramped region extends distally and radially relative to the coupler.

46.-51. (canceled)
